(19) United States
(12) Patent Application Publication
(10) Pub. No.: US 2026/0287583 A1
(43) Pub. Date: Sep. 24, 2026

(54) METHOD FOR EVALUATING RELIABILITY OF IMMUNOCHROMATOGRAPHIC TEST, TEST STRIP, AND KIT

(71) Applicant: CANON MEDICAL SYSTEMS CORPORATION, Otawara-shi (JP)

(72) Inventors: Keisuke SUGAHARA, Nasushiobara (JP); Hiroto SATAKE, Brighton, MA (US)

(73) Assignee: CANON MEDICAL SYSTEMS CORPORATION, Otawara-shi (JP)

(21) Appl. No.: 19/565,158

(22) Filed: Mar. 12, 2026

(30) Foreign Application Priority Data
Mar. 18, 2025 (JP) 2025-043644

(51) Int. Cl. G01N 33/543

(52) U.S. Cl. CPC G01N 33/54388

(57) ABSTRACT

A method for evaluating a reliability of an immunochromatographic test for detecting a measurement target molecule in a specimen according to an embodiment includes (a) introducing a specimen into an introduction region, (b) diffusing the specimen from an introduction region toward a test region, (c) detecting a molecule bound to a first capture molecule in the test region, (d) detecting a molecule bound to a second capture molecule in a check region, and (e) evaluating a reliability of an immunochromatographic test from detection results in a check region detection step. The method uses a test strip including an introduction region into which the specimen is introduced, a test region which holds the first capture molecule that binds to the measurement target molecule or a capture molecule that binds to the first capture molecule, and a check region which holds the second capture molecule that binds to a removal target molecule or a capture molecule that binds to the second capture molecule. The removal target molecule is a molecule that binds to the first capture molecule.

### CROSS-REFERENCE TO RELATED APPLICATION

The present application claims priority based on Japanese Patent Application No. 2025-043644 filed Mar. 18, 2025, the content of which is incorporated herein by reference.

### BACKGROUND OF THE INVENTION

### Field of the Invention

An embodiment disclosed in the present specification and the drawing relates to a method for evaluating a reliability of an immunochromatographic test, and a test strip and a kit, which are used for the immunochromatographic test.

### Description of Related Art

An immunochromatographic test is a test that uses a slender membrane called a strip, in which an antibody that captures a molecule to be detected is fixed in a part of the strip. A specimen is introduced to one end of the strip, diffused to the other end by a capillary phenomenon, and captured by an antibody on the strip to detect a molecule of interest. The immunochromatographic test is widely used for screening for diseases because it enables an easy and quick test.

In the immunochromatographic test, an antibody having higher specificity is selected to specifically detect a molecule of interest. In addition, it is possible to carry out quantification more easily and quickly by using a recognition antibody modified with fluorescent nanoparticles.

### BRIEF DESCRIPTION OF THE DRAWINGS

FIG. 1 is a schematic view showing an example of a configuration of a test strip according to a first embodiment.

FIG. 2 is a simplified view showing a test strip according to a second embodiment (part A), is a simplified view showing a test strip according to a third embodiment (part B), and a simplified view showing a test strip according to a fourth embodiment (part C).

FIG. 3 is a simplified view showing a test strip according to a fifth embodiment (part A) and a simplified view showing a test strip according to a sixth embodiment (part B).

### DETAILED DESCRIPTION OF THE INVENTION

Hereinafter, a method for evaluating a reliability of an immunochromatographic test according to an embodiment, a test strip, and a kit will be described with reference to the drawings. In the drawings, the same or corresponding parts are designated by the same or corresponding reference numerals, and the description thereof will not be duplicated. The dimensional ratio in each figure may be exaggerated for description and thus may not necessarily match the actual dimensional ratio.

A method for evaluating a reliability of an immunochromatographic test according to an embodiment is a method for evaluating a reliability of an immunochromatographic test for detecting a measurement target molecule in a specimen. The method according to the embodiment includes (a) introducing a specimen into an introduction region; (b) diffusing the specimen from the introduction region toward a test region after the introduction; (c) detecting a molecule bound to a first capture molecule in the test region; (d) detecting a molecule bound to a second capture molecule in a check region; and (e) evaluating a reliability of an immunochromatographic test from detection results in (d) above. The method according to the embodiment uses a test strip including an introduction region, a test region, and a check region. The introduction region is located on an upstream side of the test strip, and the specimen is introduced into the introduction region. The test region is located on a downstream side of the test strip and holds the first capture molecule that binds to the measurement target molecule or a capture molecule that binds to the first capture molecule. The check region is located between an upstream side end of the introduction region and a downstream side end of the test region and holds the second capture molecule that binds to a removal target molecule or a capture molecule that binds to the second capture molecule. The removal target molecule is a molecule that binds to the first capture molecule and is different from the measurement target molecule.

The test strip according to the embodiment is used for an immunochromatographic test for detecting the measurement target molecule in the specimen. The test strip according to the embodiment includes an introduction region, a test region, and a check region. The introduction region is located on an upstream side of the test strip, and the specimen is introduced into the introduction region. The test region is located on a downstream side of the test strip and holds the first capture molecule that binds to the measurement target molecule in the specimen or a capture molecule that binds to the first capture molecule. The check region is located between an upstream side end of the introduction region and a downstream side end of the test region and holds a second capture molecule that binds to the removal target molecule or a capture molecule that binds to the second capture molecule. The removal target molecule is a molecule that binds to the first capture molecule and is different from the measurement target molecule.

A kit according to an embodiment is used for an immunochromatographic test for detecting a measurement target molecule in a specimen. The kit according to the embodiment includes the test strip according to the embodiment and a device for removing a removal target molecule.

### <Method for Evaluating Reliability of Immunochromatographic Test>

First, the method for evaluating the reliability of the immunochromatographic test according to the present embodiment (hereinafter, may be simply referred to as the “evaluation method”).

The evaluation method according to the present embodiment is a method for evaluating a reliability of an immunochromatographic test for detecting a measurement target molecule in a specimen.

The evaluation method according to the present embodiment uses a test strip. The test strip includes the introduction region into which the specimen is introduced on an upstream side of the test strip; the test region which is located on a downstream side of the test strip and holds the first capture molecule that binds to the measurement target molecule or a capture molecule that binds to the first capture molecule; and the check region which is located between an upstream side end of the introduction region and a downstream side end of the test region and holds the second capture molecule that binds to a removal target molecule or a capture molecule that binds to the second capture molecule.

The removal target molecule is a molecule that binds to the first capture molecule and is different from the measurement target molecule.

The evaluation method according to the present embodiment includes;
(a) introducing the specimen into the introduction region (hereinafter, also referred to as the “introduction step”),
(b) diffusing the specimen from the introduction region toward the test region after the introduction step (hereinafter, also referred to as the “diffusion step”),
(c) detecting a molecule bound to the first capture molecule in the test region (hereinafter, also referred to as the “test region detection step”),
(d) detecting a molecule bound to the second capture molecule in the check region (hereinafter, also referred to as the “check region detection step”), and
(e) evaluating a reliability of an immunochromatographic test from detection results in the check region detection step (hereinafter, also referred to as the “evaluation step”).

In the immunochromatographic test in the related art, in a case where a molecule (removal target molecule) other than the measurement target molecule that binds to a molecule (first capture molecule) that captures the measurement target molecule on the test strip is present, the removal target molecule is detected in the same manner as in the case of the measurement target molecule, and thus a user cannot determine whether or not the removal target molecule has been detected, which causes a concern about the reliability of the immunochromatographic test.

In a case where the removal target molecule is sufficiently removed in the immunochromatographic test, the false positiveness is suppressed, and the amount of the measurement target molecule can be detected more accurately. In other words, in a case where the removal target molecule is sufficiently removed from the specimen, the reliability of the immunochromatographic test can be said to be high.

Therefore, in the evaluation method according to the present embodiment, by using a test strip having a check region in which the removal target molecule is detected upstream of the test region in which the measurement target molecule is detected, the amount of the removal target molecule in the specimen introduced into the test strip is examined, and the reliability of the immunochromatographic test is evaluated. By using the evaluation method according to the present embodiment, a test having a higher reliability can be carried out in the immunochromatographic test.

The test strip that is used in the evaluation method according to the present embodiment is not particularly limited as long as it is a test strip which has, on the upstream side, an introduction region for introducing a specimen, has, on the downstream side, a test region which holds the first capture molecule that binds to the measurement target molecule or a capture molecule that binds to the first capture molecule, and has a check region which is located between an upstream side end of the introduction region and a downstream side end of the test region and holds the second capture molecule that binds to a removal target molecule or a capture molecule that binds to the second capture molecule. Examples of the test strip include the test strip according to the present embodiment, which will be described later.

The measurement target molecule in the evaluation method according to the present embodiment is not particularly limited as long as it is a molecule that is detectable using an immunological reaction. The measurement target molecule may be only one kind or may be two or more kinds, and in a case where the measurement target molecules are two or more kinds, the combination and ratio thereof may be appropriately adjusted.

For the evaluation method according to the present embodiment, it is preferable to use a biomolecule that is measured in a clinical test, as a measurement target molecule. Specific examples thereof include inflammatory markers such as C-reactive protein (CRP), IgA, IgG, and IgM, fibrin degradation products (for example, D-dimer), coagulation/fibrinolysis markers such as soluble fibrin, thrombin-antithrombin complex (TAT), and plasmin-plasmin inhibitor complex (PIC), circulation-related markers such as oxidized LDL, brain natriuretic peptide (BNP), heart type fatty acid-binding protein (H-FABP), and cardiac troponin (cTn1), metabolic markers such as adiponectin, tumor markers such as carcinoembryonic antigen (CEA), alpha-fetoprotein (AFP), CA19-9, CA125, prostate-specific antigen (PSA), infectious disease-related markers such as hepatitis B virus (HBV), hepatitis C virus f (HCV), Chlamydia trachomatis, Neisseria gonorrhoeae, allergen-specific immunoglobulin E (IgE), a hormone, a drug, a nucleic acid, and the like. The nucleic acid may be, for example, a nucleic acid derived from a microorganism having pathogenic bacteria. The microorganism includes, for example, a virus, a bacterium, and the like.

In addition, various reactants after the reaction are used as a specimen, and the reaction resultant of interest in the reactants can be used as the measurement target molecules. The reaction resultant is not particularly limited and may be any of a nucleic acid, a protein, a low molecular weight compound, or the like.

The first capture molecule is not particularly limited as long as it is a molecule that is capable of being bound to the measurement target molecule. Examples of the first capture molecule include proteins such as an antibody and an enzyme; nucleic acids such as oligomers; low molecular weight compounds; and the like. Among them, an antibody is preferable from the viewpoint that it is easy to be held on the test strip.

The capture molecule that binds to the first capture molecule can be used in a case where the specimen contains a measurement target molecule that is in a state of having bound to the first capture molecule. The capture molecule that binds to the first capture molecule is not particularly limited as long as it is a molecule that is capable of being bound to the first capture molecule that is in a state of having bound to the measurement target molecule. Examples of the capture molecule that binds to the first capture molecule include proteins such as an antibody and an enzyme; nucleic acids such as oligomers; low molecular weight compounds; and the like. Among them, an antibody is preferable from the viewpoint that it is easy to be held on the test strip.

The removal target molecule is not particularly limited as long as it is a molecule other than the measurement target molecule, which binds to the first capture molecule. In the evaluation method according to the present embodiment, the removal target molecule may be only one kind or may be two or more kinds, and in a case where the measurement target molecules are two or more kinds, the combination and ratio thereof may be appropriately adjusted.

The second capture molecule is not particularly limited as long as it is a molecule different from the first capture molecule and is a molecule that can bind to the removal target molecule but does not bind to the measurement target molecule and the labeling molecule described later. Examples of the second capture molecule include proteins such as an antibody and an enzyme; nucleic acids such as oligomers; low molecular weight compounds; and the like.

The capture molecule that binds to the second capture molecule can be used in a case where the specimen contains a removal target molecule that is in a state of having bound to the second capture molecule. The capture molecule that binds to the second capture molecule is not particularly limited as long as it is a molecule that is capable of binding to the second capture molecule that is in a state of having bound to the removal target molecule but does not bind to the measurement target molecule and the labeling molecule described later. Examples of the capture molecule that binds to the second capture molecule include proteins such as an antibody and an enzyme; nucleic acids such as oligomers; low molecular weight compounds; and the like. Among them, an antibody is preferable from the viewpoint that it is easy to be held on the test strip.

Any of the bonding mode between the measurement target molecule and the first capture molecule, the bonding mode between the first capture molecule and the capture molecule that binds to the first capture molecule, the bonding mode between the removal target molecule and the second capture molecule, and the bonding mode between the second capture molecule and the capture molecule that binds to the second capture molecule is not limited to a covalent bond and may be an ionic bond, a metal bond, a hydrogen bond, a polar attractive force, a van der Waals force, or the like.

### <<Introduction Step>>

The introduction step in the evaluation method according to the present embodiment is a step of introducing a specimen into an introduction region on the test strip. The method for introduction is not particularly limited as long as it is a method in which the specimen is brought into contact with the introduction region. For example, the specimen may be dropped into the introduction region, or the introduction region may be immersed in the specimen.

### <<Diffusion Step>>

The diffusion step in the evaluation method according to the present embodiment is a step of diffusing the specimen introduced in the introduction step, toward the downstream direction from the introduction region. A specimen diffused from the introduction region to the downstream side of the test strip reaches the check region, and after or at the same time as the reaching thereof, the specimen reaches the test region. The diffusion of the specimen in the test strip is caused by a capillary phenomenon.

### <<Check Region Detection Step>>

The check region detection step in the evaluation method according to the present embodiment is a step of detecting a molecule bound to the second capture molecule in the check region among specimens diffused from the introduction region toward the test region. The check region holds the second capture molecule that binds to the removal target molecule in the specimen, or holds a capture molecule that binds to the second capture molecule. In a case where the specimen that has reached the check region contains a removal target molecule, the removal target molecule is captured by the second capture molecule held in the check region. In a case where the specimen that has reached the check region contains a removal target molecule that is in a state of having bound to the second capture molecule, a complex of the removal target molecule and the second capture molecule is captured by a capture molecule that binds to the second capture molecule held in the check region.

A molecule bound to the second capture molecule held in the check region and a molecule bound to a capture molecule that binds to the second capture molecule held in the check region can be detected by various detection methods that are utilized as detection methods in the immunochromatographic test. Examples of the detection method include a method using one of the group consisting of a coloration reaction, a chemiluminescence reaction, and a fluorescent label.

For the coloration reaction, for example, a molecule labeled with a molecule having a gold nanoparticle and bound to the second capture molecule is detected. In addition, a horseradish peroxidase (HRP)-labeled antibody that binds to the removal target molecule is used as a second capture molecule, and a complex of the removal target molecule and the HRP-labeled antibody, which is captured in the check region, can be allowed to undergo color development and then detected by a DAB method.

Examples of the chemiluminescence reaction include a reaction that uses an electrochemiluminescence method (ECL). For example, an HRP-labeled antibody that binds to a removal target molecule is used as the second capture molecule, and a complex of the removal target molecule and the HRP-labeled antibody, which is captured in the check region, can be detected by luminescence by ECL that uses luminol and an enhancer.

For a method in which a fluorescent label is used, for example, an antibody is previously labeled with a fluorescent substance, and the fluorescence emitted from the fluorescent substance used for the label is detected. As the fluorescent substance, various fluorescent substances generally used for fluorescence labeling of biomolecules, such as fluorescein and rhodamine, can be used.

The detected coloration intensity, chemiluminescence intensity, or fluorescence intensity may be visually checked and can also be detected as a signal intensity according to a publicly known image analysis. Based on the positional relationship between the check region and the test region in the test strip, the check region detection step may be carried out before the test region detection step.

### <<Test Region Detection Step>>

The test region detection step in the evaluation method according to the present embodiment is a step of detecting a molecule bound to a first capture molecule in a test region in a specimen diffused from the introduction region toward the test region. The test region holds the first capture molecule that binds to the measurement target molecule, or holds a capture molecule that binds to the first capture molecule. In a case where the specimen that has reached the test region contains a measurement target molecule, the measurement target molecule is captured by the first capture molecule held in the test region. In a case where the specimen that has reached the test region contains a measurement target molecule that is in a state of having bound to the first capture molecule, a complex of the measurement target molecule and the first capture molecule is captured by a capture molecule that binds to the first capture molecule held in the test region.

Examples of the detection method for the molecule bound to the first capture molecule include the same one as the detection method for the molecule bound to the second capture molecule in the check region. The detection method for the molecule bound to the first capture molecule in the check region and the detection method for the molecule bound to the second capture molecule in the check region may be the same method or may be methods different from each other.

### <<Evaluation Step>>

The evaluation step in the evaluation method according to the present embodiment is a step of evaluating the reliability of the immunochromatographic test from the detection results in the check region detection step.

In the check region, the fact that the molecule bound to the second capture molecule is not detected or only a very small amount thereof is detected indicates that the specimen that has reached the test region does not contain the removal target molecule, or only a very small amount of the removal target molecule is contained. Therefore, in a case where the molecule bound to the second capture molecule is not detected in the check region, it can be evaluated that the reliability of the immunochromatographic test is high. Alternatively, in a case where the detection amount of the molecule bound to the second capture molecule is less than a predetermined threshold value, it can be evaluated that the reliability of the immunochromatographic test is high. The threshold value can be experimentally determined in advance by using a simulated specimen containing a concentration-known removal target molecule and a measurement target molecule.

### <<Other Steps>

### (Removal Step)

The evaluation method according to the present embodiment may have a removal step of removing a removal target molecule in the specimen before the introduction step. The removal step is not particularly limited as long as the removal target molecule is removed while the measurement target molecule is not removed, and the removal target molecule can be removed using filtration, adsorption, aggregation, or the like.

For example, filter filtration may be carried out to remove the removal target molecule. The removal target molecule and the measurement target molecule have the same site to which the first capture molecule is bound. Therefore, in the filter filtration, a filter capable of removing the measurement target molecule from the specimen by utilizing a site other than the same site described above is used. More specifically, it is possible to apply to a filter or affinity column, in which the second capture molecule is held before the specimen is introduced into the test strip.

Alternatively, a filter region may be provided in the test strip instead of the removal step. While the specimen is diffused from the introduction region to the front of the check region, the removal target molecule in the specimen may be captured in a filter region which holds a third capture molecule that binds to the removal target molecule.

The third capture molecule is not particularly limited as long as it is a molecule that is different from the first capture molecule and is capable of binding to the removal target molecule. Examples of the third capture molecule include the same ones as the second capture molecule, and the third capture molecule may be the same as or different from the second capture molecule.

The bonding mode between the removal target molecule and the third capture molecule, and the bonding mode between the third capture molecule and the molecule that binds to the third capture molecule include the same bonding modes as the bonding modes between the above-described removal target molecule and the second capture molecule, and the like.

### (Labeling Step)

The evaluation method according to the present embodiment may have a labeling step in which the measurement target molecule and the removal target molecule in the specimen are labeled with a labeling molecule before the introduction step. Alternatively, while the specimen is diffused from the introduction region to the front of the check region, the measurement target molecule and the removal target molecule in the specimen may be labeled with a labeling molecule.

The labeling molecule is a molecule that binds to the measurement target molecule or the removal target molecule, for detection of a complex of the measurement target molecule and the first capture molecule, a complex of the measurement target molecule, the first capture molecule, and the capture molecule that binds to the first capture molecule, a complex of the removal target molecule and the second capture molecule, or a complex of the removal target molecule, the second capture molecule, and the capture molecule that binds to the second capture molecule. By binding the labeling molecules to these, the removal target molecule in the check region or the measurement target molecule in the test region can be detected. The labeling molecules that are used to label the measurement target molecule and the removal target molecule may be the same or may be different from each other.

The labeling molecule can be appropriately selected and used in accordance with the detection method for each complex. Specific examples of the labeling molecule include a molecule having a fluorescent substance, a luminescent substrate, a magnetic material, an enzyme, a gold nanoparticle, and the like.

Examples of the fluorescent substance include green fluorescent protein (GFP), red fluorescent protein (RFP), and fluorescein (FAM), and the like.

Examples of the luminescent substrate include luminol, a ruthenium complex, and the like.

Examples of the magnetic material include iron, cobalt, nickel, oxides of iron, cobalt, nickel, a ferrite, alloys thereof, and the like.

Examples of the enzyme include peroxidase, alkaline phosphatase, galactosidase, β-glucuronidase, and the like.

Examples of the shape of the gold nanoparticles include a spherical shape, a spike shape, a rod shape, and the like.

The bonding mode between the labeling molecule and the other molecule is not limited to the covalent bond but may be an ionic bond, a metal bond, a hydrogen bond, a polar attractive force, a van der Waals force, or the like.

The labeling molecule may be the same as or may be different from any one of the first capture molecule, the capture molecule that binds to the first capture molecule, the second capture molecule, or the capture molecule that binds to the second capture molecule. For example, in a case where a molecule obtained by subjecting an antibody against the measurement target molecule to fluorescence labeling is used as the first capture molecule, or in a case where a molecule obtained by subjecting an antibody against the removal target molecule to fluorescence labeling is used as the second capture molecule, the first capture molecule or the second capture molecule serves as a labeling molecule.

Regarding the labeling step, specifically, the removal target molecule in the specimen is removed by a method such as filtration, adsorption, aggregation, or the like, and then the labeling molecule is added to the specimen to label the removal target molecule and the measurement target molecule with the labeling molecule. In addition, the labeling molecule may be added to the specimen to label the removal target molecule and the measurement target molecule with the labeling molecule, and then the removal target molecule in the specimen may be removed.

### (Control Region Detection Step)

In the evaluation method according to the present embodiment, in a case where the test strip has, downstream of the test region, a control region which holds the fourth capture molecule that binds to any component contained in the specimen that has passed through the test region, the specimen may be diffused from the introduction region to the control region. Further, a control region detection step of detecting a molecule bound to the fourth capture molecule in the control region may be provided after the diffusion. In a case where the control region detection step is provided, it can be seen that the specimen has flowed to the downstream part, that is, the test has been established.

The fourth capture molecule is not particularly limited as long as it is a molecule different from the first to third capture molecules and is a molecule that does not bind to the measurement target molecule and the removal target molecule. Examples of the fourth capture molecule include proteins such as an antibody and an enzyme; nucleic acids such as oligomers; low molecular weight compounds; and the like. Among them, an antibody is preferable from the viewpoint that it is easy to be held on the test strip. In the evaluation method according to the present embodiment, in a case where a labeling molecule independent of the first capture molecule and the second capture molecule is used, the labeling molecule can be used as the fourth capture molecule.

The bonding mode between the fourth capture molecule and the molecule that binds to the fourth capture molecule includes the same bonding modes as those for the first and second capture molecules described above.

The detection of the molecule bound to the fourth capture molecule can be carried out in the same manner as the method described in the check region detection step.

For example, in a case where a molecule bound to the second capture molecule is not detected in the check region, a molecule bound to the first capture molecule is detected in the test region, and a molecule bound to the fourth capture molecule is detected in the control region, the result of the immunochromatographic test is positive, and it can be evaluated that the result has high reliability.

In addition, in a case where a molecule bound to the second capture molecule is not detected in the check region, a molecule bound to the first capture molecule is not detected in the test region, and a molecule bound to the fourth capture molecule is detected in the control region, the result of the immunochromatographic test is negative, and it can be evaluated that the result has high reliability.

In a case where a molecule bound to the second capture molecule is detected in the check region, a molecule bound to the first capture molecule is detected in the test region, and a molecule bound to the fourth capture molecule is detected in the control region, although the result of the immunochromatographic test is positive, the reliability is low, and it may be evaluated the result is false positive or the test is rejected.

Alternatively, in a case where the molecule bound to the fourth capture molecule is not detected in the control region, the result of the immunochromatographic test may be evaluated such that the test is rejected.

The specimen that is used in the present embodiment is not particularly limited as long as it can be used for the immunochromatographic test, and the specimen may be a biological specimen such as blood, urine, sputum, saliva, nasal discharge, nasal swab, pharyngeal swab, other body fluids, or feces. The biological specimen may be used as a specimen as it is, and the biological specimen includes a biological specimen that has undergone a reaction, dilution, extraction, or separation. Examples of the blood specimen include red blood cells, blood plasma, serum, and the like, in addition to whole blood. The specimen may contain a measurement target molecule and a removal target molecule.

Alternatively, the specimen may be mixed with a buffer to adjust the pH, viscosity, or the like. As an example, 100 μL of a Tris Buffered Saline-based buffer (HybriDetect Assay Buffer, #MGCB, Milenia Biotec GmbH) may be added to 10 μL of a sample.

<Test Strip that is Used for Immunochromatographic Test>

The test strip that is used for the immunochromatographic test of the present embodiment (hereinafter, may be simply referred to as the “test strip according to the present embodiment”) is used for an immunochromatographic test for detecting a measurement target molecule in a specimen. The test strip according to the present embodiment includes the introduction region which is located on an upstream side of the test strip and into which the specimen is introduced; the test region which is located on a downstream side of the test strip and holds the first capture molecule that binds to the measurement target molecule or a capture molecule that binds to the first capture molecule; and the check region which is located between an upstream side end of the introduction region and a downstream side end of the test region and holds the second capture molecule that binds to a removal target molecule or a capture molecule that binds to the second capture molecule. The removal target molecule is a molecule that binds to the first capture molecule and is different from the measurement target molecule.

In the test strip according to the present embodiment, the amount of the removal target molecule in the specimen that has reached the test region in the immunochromatographic test is examined by providing a check region in which the removal target molecule is detected upstream of the test region in which the measurement target molecule is detected, and the reliability of the immunochromatographic test can be evaluated.

The test strip according to the present embodiment may be a test strip shown in any of the first to sixth embodiments, which is not limited thereto.

Hereinafter, one embodiment of the test strip will be described with reference to the drawing. It is noted that, in each view, for the same portion as the portion in the first embodiment and a portion not related to the explanatory description, the illustration and an explanatory description thereof may be omitted or simplified.

### First Embodiment

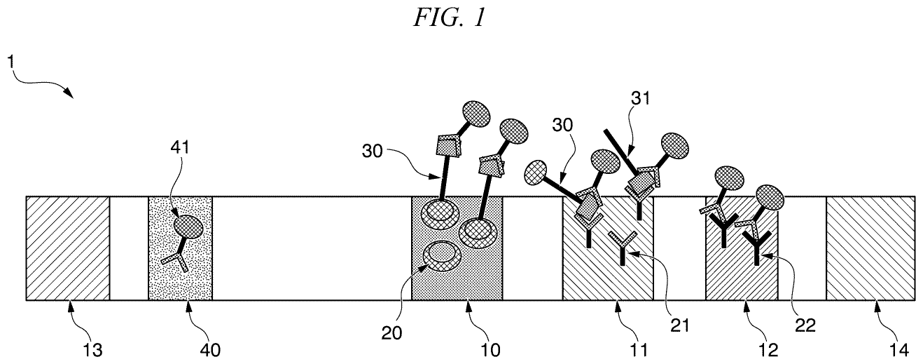

FIG. 1 is a view showing an example of a configuration of a test strip according to a first embodiment.

The test strip 1 has on the upstream side, for example, an introduction region 13 into which a specimen is introduced, has, on the downstream side, a test region 11 which holds a first capture molecule 21 that binds to a measurement target molecule 31, and has a check region 10 which holds a second capture molecule 20 that binds to a removal target molecule 30, between the upstream side end of the introduction region 13 and the downstream side end of the test region 11, where the removal target molecule 30 is a molecule that binds to the first capture molecule 21.

Further, each of a labeling region 40 containing a labeling molecule 41, a control region 12 for detecting the labeling molecule 41, and a water absorption region 14 for absorbing the specimen solution may be provided.

For the main material of the test strip 1, for example, a material used in the immunochromatographic test in the related art is used. A main material of the test strip 1 is, for example, nitrocellulose or the like. In addition, as an example of the size of the test strip 1, a test strip having a length of 50 to 100 mm and a width of 2 to 10 mm can be used.

### (Introduction Region)

The introduction region 13 is a region in which a specimen in contact with the surface of the region is absorbed into the inside of the test strip and introduced. A specimen introduced into the introduction region is diffused by a capillary phenomenon and then allowed to be fed downstream of the test strip.

For the material of the introduction region 13, for example, a cellulose fiber filter or the like can be used in the same way as in the immunochromatographic test in the related art.

### (Test Region)

The test region 11 holds the first capture molecule 21 that binds to the measurement target molecule 31. The test region 11 is a region for detecting the measurement target molecule, and it shows the results of the immunochromatographic test. The test region 11 may be used to evaluate whether or not the specimen has flowed downstream from the check region 10 in a case where the reliability of the immunochromatographic test is evaluated.

Examples of the first capture molecule 21 that binds to the measurement target molecule include the same one as the first capture molecule that is used in the evaluation method according to the present embodiment.

### (Check Region)

The check region 10 is present between the upstream side end of the introduction region and the downstream side end of the test region, and it holds the second capture molecule 20 that binds to the removal target molecule 30. The check region 10 is a region for detecting the removal target molecule in the specimen that has reached the check region, and it is used to evaluate the reliability of the immunochromatographic test.

Examples of the second capture molecule 20 that binds to the removal target molecule 30 include the same one as the second capture molecule that is used in the evaluation method according to the present embodiment.

### (Labeling Region)

The test strip 1 may have the labeling region 40 between the upstream side end of the introduction region 13 and the upstream side end of the check region 10. The labeling region 40 contains a labeling molecule 41 for labeling the removal target molecule 30 and the measurement target molecule 31.

Examples of the labeling molecule 41 include the same one as the labeling molecule that is used in the evaluation method according to the present embodiment. The labeling molecule 41 is, for example, an anti-HRP antibody labeled with gold nanoparticles, an antibody labeled with gold nanoparticles, or the like.

Since the check region 10 and the test region 11 are spatially independent, the labeling molecule 41 that is bound to the removal target molecule 30 and the labeling molecule 41 that is bound to the measurement target molecule 31 may be the same or may be different from each other.

For the material of the labeling region 40, for example, glass fibers or the like, which is used in the immunochromatographic test in the related art, can be used.

In a case where the removal target molecule 30 and the measurement target molecule 31 in the specimen are labeled with the labeling molecule 41 before the specimen is introduced into the introduction region 13, the labeling region 40 may not be present.

### (Control Region)

The control region 12 holds a fourth capture molecule 22. The control region 12 is used for evaluating whether or not the specimen has flowed to the downstream part of the test region 11 in the immunochromatographic test. The control region can be omitted.

Examples of the fourth capture molecule 22 include the same one as the fourth capture molecule that is used in the evaluation method according to the present embodiment.

### (Water Absorption Region)

The water absorption region 14 absorbs the specimen solution that has flowed. In a case where the test strip 1 has the water absorption region 14, the water absorption region 14 is provided at the most downstream end of the test strip 1. For the material of the water absorption region 14, for example, cellulose or the like, which is used in the immunochromatographic test in the related art, can be used.

### Second Embodiment

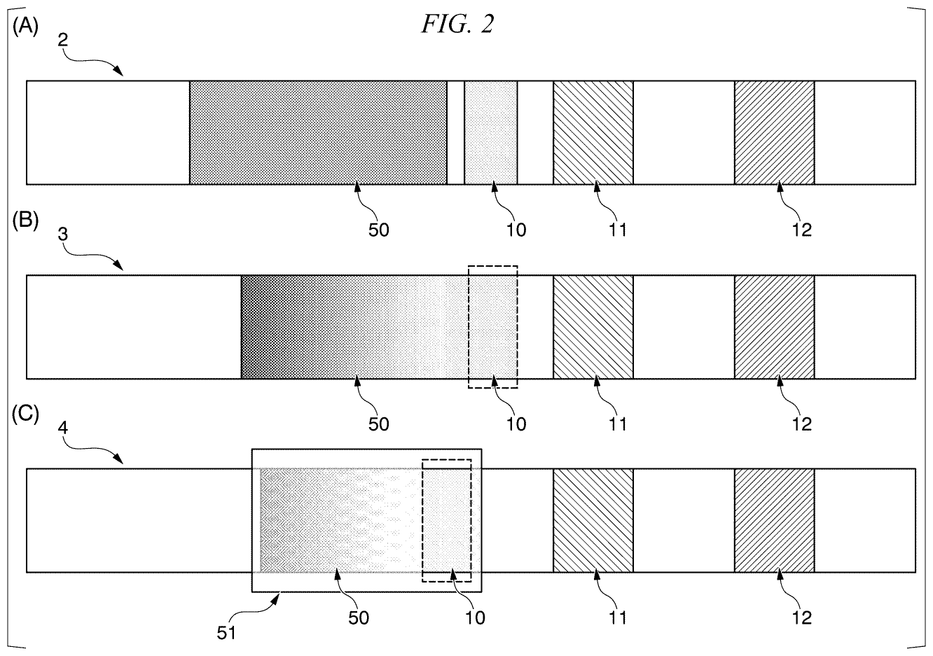

As shown in FIG. 2 (part A), the test strip 2 according to the second embodiment has a filter region 50 that holds a third capture molecule that binds to the removal target molecule 30 upstream of the check region 10. It suffices that the test strip 2 may have the filter region 50 between the upstream side end of the introduction region 13 and the downstream side end of the check region 10. It is noted that although not illustrated in the drawing, the test strip 2 has the introduction region 13. In addition, the test strip 2 may have or may not have each of the labeling region 40 and the water absorption region 14.

Since the test strip 2 has the filter region 50, the test strip 2 can be used for evaluating the reliability of the immunochromatographic test without removing the removal target molecule in the specimen in advance. In addition, since the filter region 50 and the check region 10 are provided to be spatially independent, the detection can be carried out in the check region 10 without being affected by the filter region 50.

Examples of the third capture molecule that binds to the removal target molecule 30 include the same one as the second capture molecule 20 that binds to the removal target molecule 30, which may be identical. That is, the filter region 50 may hold the second capture molecule 20. In this case, the filter region 50 holds more second capture molecules 20 than the check region 10, thereby sufficiently reducing the amount of the removal target molecule in the specimen that reaches the check region, and it is easy to evaluate that the immunochromatographic test is highly reliable.

It suffices that the filter region 50 has a sufficient amount of the third capture molecule. Specifically, the filter region 50 can be designed as follows.

In general, the test strip that is used for the immunochromatographic test is porous, and thus the surface area (internal surface area) considering the internal porous structure is large as compared with the apparent surface area. In general, the ratio of the internal surface area to the apparent surface area is about 50 to 200. The mass of the protein that can be immobilized on the test strip is approximately determined by the size and the like of the protein, and the mass of the immobilized protein in the apparent surface area (surface protein capacity: Cp) can be calculated by multiplying the ratio of the internal surface area to the apparent surface area by the mass of the protein that can be immobilized on the test strip. Based on the calculated Cp, the filter region can be designed so that a sufficient amount of the third capture molecule can be ensured on the test strip with respect to the removal target molecule 30.

More specifically, in a case where the filter region 50 having X times the third capture molecule with respect to the material quantity (B) of the introduced removal target molecule 30 is designed, the area(S) of the filter region can be designed from the following expression, where the binding rate between the removal target molecule 30 and the third capture molecule is denoted as R.

$$S = BX / CpR$$

In addition, in a case where a filter region having a length L is provided in a test strip having a width W, L is indicated by the following expression.

$$L = S / W$$

### Third Embodiment

As shown in FIG. 2 (part B), the test strip 3 according to the third embodiment has the same configuration as that of the test strip 2; however, the filter region 50 and the check region 10 are adjacent to each other. The concentration of the third capture molecule held in the filter region 50 may be uniform from the introduction region side toward the check region side, or may decrease from the introduction region side toward the check region side. In a case where the concentration of the third capture molecule decreases from the introduction region side toward the check region side, the detection can be carried out in the check region 10 without being affected by the filter region 50.

### Fourth Embodiment

As shown in FIG. 2 (part C), the test strip 4 according to the fourth embodiment has the same configuration as that of the test strip 3 according to the third embodiment, and further has a covering member 51 that covers the filter region 50. The covering member 51 does not cover the check region 10. The material of the covering member 51 is not particularly limited as long as the filter region 50 can be made difficult to be visually recognized. In the test strip 3, since the filter region 50 is covered with a covering member 51, it is easier to visually distinguish the filter region 50 from the check region 10.

### Fifth Embodiment

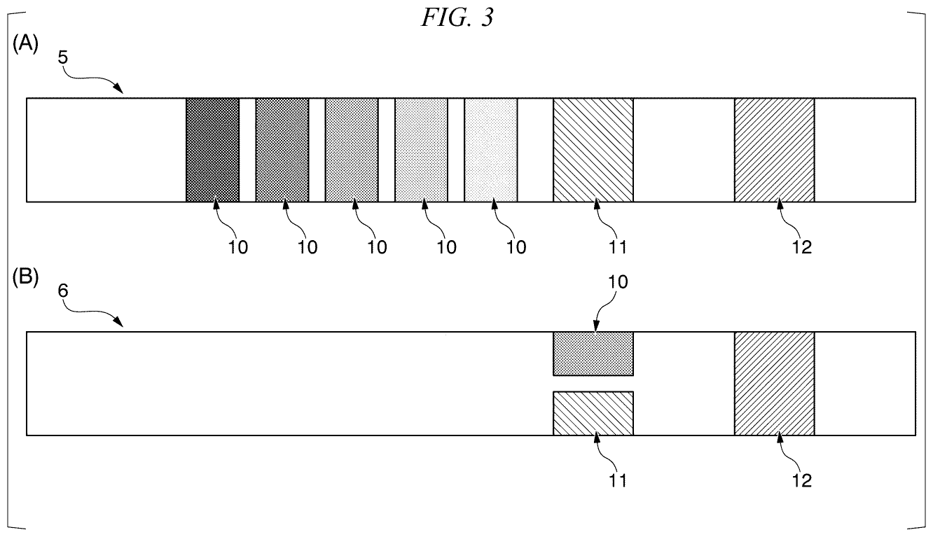

As shown in FIG. 3 (part A), the test strip 5 according to the fifth embodiment has a plurality of check regions 10. Since the test strip 5 has two or more check regions 10, it is possible to stepwise evaluate the detection of the molecule bound to the second capture molecule 20 in the check region 10.

### Sixth Embodiment

As shown in FIG. 3 (part B), the test strip 6 according to the sixth embodiment has the check region 10 and the test region 11 in parallel with the flow direction of the specimen. In the test strip 6, it is possible to detect a molecule bound to the first capture molecule 21 in the test region 11 without the test region 11 being affected by the molecule bound to the second capture molecule in the check region 10.

<Kit that is Used for Immunochromatographic Test>

A kit that is used for the immunochromatographic test according to the present embodiment is used for an immunochromatographic test for detecting a measurement target molecule in a specimen. The kit according to the present embodiment includes the test strip according to the embodiment and a device for removing a removal target molecule.

The device for removing the removal target molecule is not particularly limited as long as it is a device that does not remove the measurement target molecule while removing the removal target molecule, and it may be any device that can remove the removal target molecule using filtration, adsorption, aggregation, or the like. For example, a filter or an affinity column, which can remove the removal target molecule from the specimen by utilizing a site other than the same site possessed by the removal target molecule and the measurement target molecule, may be provided. More specifically, a filter or an affinity column, in which the second capture molecule is held, may be provided. The device may be a specimen dropping device having a filter or an affinity column.

### EXAMPLES

Hereinafter, the present invention will be described in more detail based on Examples; however, the present invention is not limited to these Examples.

### Example 1

The CRISPR-Cas reaction is a reaction that recognizes a specific base sequence (target sequence) and cleaves nucleic acids. A target nucleic acid can be cleaved by adding a Cas enzyme and an sgRNA having a sequence complementary to a target sequence to a nucleic acid (target nucleic acid) having the target sequence. In the immunochromatographic test, the presence or absence of the cleavage of the target nucleic acid can be evaluated by subjecting a reaction solution after the reaction to the test strip to detect the cleaved fragment. However, it is difficult to carry out detection by distinguishing the unreacted substance (target nucleic acid before cleavage) from the cleaved fragment, and in a case where the unreacted substance is not sufficiently removed from the reaction solution, the result of the immunochromatographic test may be false positive, and the reliability is low. Therefore, a specimen obtained by removing a target nucleic acid (unreacted substance) from the specimen is provided for the immunochromatographic test.

By using a cleaved fragment of a target nucleic acid as the measurement target molecule, using the target nucleic acid as the removal target molecule, and using the evaluation method according to the present embodiment, it is possible to evaluate the reliability in the immunochromatographic test in which the cleaved fragment of the nucleic acid by the CRISPR-Cas reaction is detected.

### (Specimen)

In a case of having carried out a CRISPR-Cas reaction using a single-stranded DNA in which the 5′-terminal is modified with FAM and the 3′-terminal is modified with biotin-labeled, a reaction solution after the reaction contains an unreacted target nucleic acid (FAM/biotin-labeled ssDNA), as well as a single-stranded DNA (FAM-labeled ssDNA) having a 5′-terminal labeled with FAM and a single-stranded DNA having a 3′-terminal labeled with biotin (biotin-labeled ssDNA), which are cleaved fragments. Therefore, in the present example, an immunochromatographic test was carried out using a simulated reaction solution as a specimen, in which the FAM-labeled ssDNA was used as a measurement target molecule and the FAM/biotin-labeled ssDNA was used as a removal target molecule. The composition of each specimen is shown in Table 1.

### (Test Strip)

In the present example, a test strip having a labeling region, a check region, and a test region in order toward the downstream with an introduction region on the uppermost stream side was used. It is noted that an anti-FAM antibody (manufactured by Milenia Biotec) labeled with gold nanoparticles was contained in the labeling region as a labeling molecule. In the check region, streptavidin (manufactured by Milenia Biotec) was held as a second capture molecule that binds to the removal target molecule. In the test region, an anti-rabbit IgG antibody (manufactured by Milenia Biotec) was held as a first capture molecule that binds to the measurement target molecule.

### (Evaluation of Reliability in Immunochromatographic Test)

10 μL of the specimen was dropped into the introduction region on the test strip, and the dropped specimen was diffused from the introduction region toward the test region. FAM/biotin-labeled ssDNA in the specimen was labeled with an anti-FAM antibody in the labeling region while the specimen was diffused from the introduction region to the front of the check region. Five minutes after the dropping, FAM/biotin-labeled ssDNA bound to the streptavidin in the check region and FAM-labeled ssDNA bound to the anti-rabbit IgG antibody in the test region were detected using coloration of gold nanoparticles by an Au-labeled anti-FAM antibody. From the detection results, the reliability of the immunochromatographic test was evaluated. The results are shown in Table 1. It is noted that the coloration of the test region was confirmed in all specimens.

TABLE 1

| | Example | Example | Example | Example |
|---|---|---|---|---|
| | 1-1 | 1-2 | 1-3 | 1-4 |
| Removal target | Final | Final | — | — |
| molecule | concentra- | concentra- | | |
| | tion: 8 μM | tion: 8 μM | | |
| Measurement | Final | — | Final | — |
| target | concentra- | | concentra- | |
| molecule | tion: 100 nM | | tion: 100 nM | |
| Coloration of | Colored | Colored | Not | Not |
| check region | | | colored | colored |

Example 1-1 is a specimen for imitating the removal treatment for the unreacted target nucleic acid (FAM/biotin-labeled ssDNA) in the reaction solution of the CRISPR-Cas reaction, Example 1-2 is a specimen for imitating a liquid before the reaction, and Example 1-3 is a specimen for imitating a liquid after the removal treatment for the unreacted target nucleic acid. Example 1-4 is a negative control that does not contain any of the target molecules. As shown in Table 1, the check region was colored in Example 1-1 and Example 1-2, each of which contained the FAM/biotin-labeled ssDNA that was the removal target molecule. On the other hand, the check region was not colored in Examples 1-3 and 1-4, each of which did not contain the FAM/biotin-labeled SSDNA.

From these facts, it was revealed that the reliability of the immunochromatographic test can be evaluated by using the evaluation method according to the present embodiment.

While some embodiments have been described, these embodiments are presented as examples and are not intended to limit the scope of the invention. These embodiments may be implemented in various other forms, and various omissions, replacements, and changes may be made to the extent that they do not deviate from the gist of the invention. These embodiments and the modification thereof are included in the scope and gist of the invention and also included in the scope of the invention described in the appended claims and the scope of its equivalents.

What is claimed is:

1. A method for evaluating a reliability of an immunochromatographic test for detecting a measurement target molecule in a specimen, the method comprising:
(a) introducing a specimen into an introduction region;
(b) diffusing the specimen from the introduction region toward a test region after the introduction;
(c) detecting a molecule bound to a first capture molecule in the test region;
(d) detecting a molecule bound to a second capture molecule in a check region; and
(e) evaluating a reliability of an immunochromatographic test from detection results in (d) above,
wherein the method uses a test strip,
the test strip includes:
the introduction region which is located on an upstream side of the test strip and into which the specimen is introduced,
the test region which is located on a downstream side of the test strip and holds the first capture molecule that binds to the measurement target molecule or a capture molecule that binds to the first capture molecule, and
the check region which is located between an upstream side end of the introduction region and a downstream side end of the test region and holds the second capture molecule that binds to a removal target molecule or a capture molecule that binds to the second capture molecule, and
the removal target molecule is a molecule that binds to the first capture molecule and is different from the measurement target molecule.

2. The method according to claim 1,
wherein in a case where the molecule bound to the second capture molecule is not detected, the reliability of the immunochromatographic test is evaluated to be high in (e) above.

3. The method according to claim 1,
wherein in a case where a detection amount of the molecule bound to the second capture molecule is less than a predetermined threshold value, the reliability of the immunochromatographic test is evaluated to be high in (e) above.

4. The method according to claim 1, further comprising:
removing the removal target molecule in the specimen before (a) above.

5. The method according to claim 1,
wherein while the specimen is diffused from the introduction region to a front of the check region, the removal target molecule in the specimen is captured in a filter region which holds a third capture molecule that binds to the removal target molecule.

6. The method according to claim 4,
wherein the removal of the removal target molecule is carried out by using the second capture molecule.

7. The method according to claim 1, further comprising:
labeling the removal target molecule in the specimen with a labeling molecule before (a) above.

8. The method according to claim 1,
wherein while the specimen is diffused from the introduction region to a front of the check region, the removal target molecule in the specimen is labeled in a labeling region containing a labeling molecule that binds to the removal target molecule.

9. The method according to claim 1,
wherein a molecule bound to the second capture molecule is detected by using one of the group consisting of a coloration reaction, a chemiluminescence reaction, and a fluorescent label.

10. The method according to claim 1,
wherein the test strip has a control region on the downstream side of the test region, and
the specimen is diffused from the introduction region to the control region.

11. A test strip that is used for an immunochromatographic test for detecting a measurement target molecule in a specimen, the test strip comprising:
an introduction region which is located on an upstream side of the test strip and into which the specimen is introduced;
a test region which is located on a downstream side of the test strip and holds a first capture molecule that binds to the measurement target molecule; and
a check region which is located between an upstream side end of the introduction region and a downstream side end of the test region and holds a second capture molecule that binds to the removal target molecule,
wherein the removal target molecule is a molecule that binds to the first capture molecule and is different from the measurement target molecule.

12. The test strip according to claim 11,
wherein the specimen subjected to a removal treatment of the removal target molecule is introduced into the introduction region.

13. The test strip according to claim 11, further comprising:
a filter region which holds a third capture molecule that binds to the removal target molecule, between the upstream side end of the introduction region and an upstream side end of the check region.

14. The test strip according to claim 13,
wherein the filter region holds the second capture molecule.

15. The test strip according to claim 11,
wherein two or more check regions are present.

16. The test strip according to claim 13,
wherein the filter region and the check region are provided to be adjacent to each other.

17. The test strip according to claim 16,
wherein the filter region is covered with a covering member.

18. A kit comprising:
the test strip according to claim 11; and
a device for removing the removal target molecule,
wherein the kit is used for an immunochromatographic test for detecting the measurement target molecule in the specimen.
